(19) United States
(12) Patent Application Publication
(10) Pub. No.: US 2005/0009858 A1
(43) Pub. Date: Jan. 13, 2005

(54) Imiquimod therapies

(76) Inventors: Maria I Martinez-Colon, San Juan, PR (US); Ignacio Sanchez Carpintero, Navarra (ES); Martin C. Mihm JR., Brookline, MA (US); Paula E. Morth, Little Rock, AR (US)

Correspondence Address: PATENT LAW OFFICES OF HEATH W. HOGLUND, 256 ELEANOR ROOSEVELT STREET, SAN JUAN, PR, 00918, US

(21) Appl. No.: 10/495,541

(22) PCT Filed: Nov. 18, 2002
(86) PCT No.: PCT/US02/37106

Related U.S. Application Data
(60) Provisional application No. 60/332,454, filed on Nov. 17, 2001.
(60) Provisional application No. 60/392,222, filed on Jun. 27, 2002.

(51) Int. Cl. A61K 31/4745

(52) U.S. Cl. 514/292

(57) ABSTRACT

Imiquimod is used to treat lichen sclerosus and vascular tumors including infantile hemangiomas. Specific applications related to lichen sclerosus are detailed first, followed by specific applications related to infantile hemangiomas.

### I. FIELD OF THE INVENTION

The invention relates to the use of Imiquimod to treat Lichen Sclerosus and vascular tumors including infantile hemangiomas. Applications related to Lichen Sclerosus are discussed first, followed by applications related to infantile hemangiomas.

### II. BACKGROUND OF LICHEN SCLEROSUS

Lichen Sclerosus (LS) is a skin disease of poorly understood etiology that occurs most commonly on the vulva and penis but can affect other areas of the skin. While the management of LS has improved with the effectiveness of ultrapotent topical steroids, it still remains a therapeutic challenge.

Further background is provided by the following references each of which are incorporated herein by reference.
1. Martinez et al. Arch Dermatol 2002; 138 (in press)
2. Suzuki et al. J Invest Dermatol 2000; 114:139-41
3. Carli P et al. Dermatologica 1991;182:18-22
4. Carli P et al. J Reprod Med 1994;39:110-104
5. Gross T et al. Dermatology 2001;202:198-202
6. Shimizu M et al. Arch Dermatol Res 1997; 289:527-32
7. Regaur et al. Am J Pathol 2002;160:103545
8. Carli et al. J Reprod Med 1997;42:161-5
9. Sauder D N. J Am Acad Dermatol 2000;43:S6-11.
10. Dahl M V. J Am Acad Dermatol 2000;43:S1-5.
11. Hengge U R et al. Lancet Infect Dis 2002; 1 (3): 189-98

These references are referred to in the following section (Section II) by their respective reference numeral.

### III. DETAILED DESCRIPTION OF INVENTION APPLIED TO LICHEN SCLEROSUS

Topical application of imiquimod, an immunomodulatory drug, has been used to treat diseases such as genital warts, superficial squamous cell carcinoma and most recently infantile hemangiomas¹ (See also poster # 1704). Because LS has an inflammatory component, we treated a patient with penile LS with topical imiquimod cream for two weeks with complete disappearance of the lesions.

A. Clinical Case

A 30 year old male presented to our office (MIM) with the main complaint of whitish patches on the penis of 2 months duration associated with occasional pruritus. Prior treatment by other physicians with topical antifungal agents and topical corticosteroids had failed. After an initial examination by one of us (MIM), a biopsy of a lesion was performed and revealed inflammatory lichen sclerosus. Basic laboratory investigations including complete blood cell count, urinalysis, blood chemistry profile, and testing for HIV revealed no abnormalities.

Because of its immunomodulatory effects, treatment of the LS lesions with imiquimod 5% cream was offered to the patient. It was clearly explained to him that this was an off label use of the medication. He began topical application to the affected areas every other day.

B. Results

A strong reaction at the treated areas with pruritus, burning and intermittently mild pain developed after 2 weeks. The affected sites exhibited erythematous, edematous plaques that were well defined and did not extend onto uninvolved skin. The lesions were red, edematous and glistening. At this time imiquimod application was stopped and the patient was started on Domeboro cold compresses to alleviate the symptoms.

Two weeks later all the lesions of LS that were treated and developed the reaction had disappeared leaving no signs of any cutaneous change. Only one small lesion on the ventral surface of the glans persisted, but this area had been left untreated by the patient received no application of the Imiquimod cream, because he had forgotten about its presence.

C. Histopathology

The biopsy of the test sites revealed in routinely processed and stained skin mild epidermal atrophy with a lichenoid inflammatory infiltrate predominantly of lymphocytes. A mild hyalinized fibrosis of the superficial, inflamed dermis was present. CD4 and CD8 stains showed a relative increase in cytotoxic T cells in the epithelium and at its interface. Scattered CD57 cells were observed in the dermal infiltrate and represented 5% of the lichenoid host response. CD1a stain revealed striking increase in dendritic cells (CD1a positive) in the lower epidermis and in the inflamed papillary dermis.

D. Discussion

Even though the cause of LS is not known and its pathogenesis is not completely characterized, various studies have implicated an immune basis for this disorder. It has been postulated that the primary event involves the activation of Langerhans cells in the dermis and epidermis.² Immumohistochemical studies^{3,4} have shown that the infiltrate is composed mainly of T cells that are activated (HLA-Dr+) and that these cells are associated with CD1a+ antigen presenting cells. Increased numbers of the latter cells were found in the dermal infiltrate and in the epidermis. Further studies have indicated that the T cells express TIA-1 (T-cell restricted intracellular antigen) and the activated T cell forms express Granzyme B.⁵ Interestingly, the latter cells were shown to be particularly associated with basal keratinocytes, suggesting that hydropic degeneration in LS is at least in part due to a cellular-dependent mechanism. Colloid bodies (Civatte in lichen planus) have been shown to be the result of cytotoxic effect with subsequent apoptosis.⁶ The finding of monoclonality in the T cells of LS suggests a direct response to an antigen in the disorder with clonal proliferation.⁷ Furthermore, these activated cells release cytoldnes that culminate in collagen degeneration and fibrosis. Thus, a greater expression of fibrogenic cytokines (IL-4, IL-6, and TGF-β) has been described in the dermis of LS.⁸.

Imiquimod is an immune response modifier, affecting both the innate and acquired immune response. Imiquimod principally affects innate immunity and achieves its effect through the production of a large number of cytokines including interferon-alpha (IFN-□), interleukin-6 (IL-6), tumor necrosis factor (TNF) as well as Granulocyte colony-stimulating factor (G-CSF), and Granulocyte-Macrophage colony stimulating factor (GM-CSF). Other interleukins IL-1, 5, 8, 10, and 12, Macrophage inflammatory protein (MIP-1), and macrophage chemotatic protein (MCP-1) are produced. It has also been reported to increase natural killer cell activity and stimulates B-cell proliferation and maturation.^{9,10} A recent study indicates that imiquimod acts as CpG-sequences that stimulate innate immunity.¹¹

As far as acquired immunity is concerned, there is evidence that topical Imiquimod increases both Langerhans cell antigen presentation and migration of Langerhans cells to regional draining lymph nodes. It also results in production of IFN-α, and IL-12. The last results from the activation of Th1.

How Imiquimod resulted in the clearance of our patients LS lesions is not clear. We think it may have acted through the stimulation of the innate and acquired immunity of the patient, causing a release of cytokines of both Th1 and Th2 origin. As in all systems a delicate balance exists. In this instance both fibrogenic proteins are released along with fibrogenic inhibitors such as IL-12 that leads to the release of IFN-γ. Apparently, this latter effect predominates in inflammatory LS with the resolution of the lesions.

E. Conclusions

Topical Imiquimod treatment offers new hope with minimal side-effects for this often refractory genital disease.

### IV. BACKGROUND OF INFANTILE HEMANGIOMAS (IH)

IH is a distinct category of benign vascular tumor characterized by presentation within the first few weeks of life, rapid growth during the first year and a subsequent variable degree of spontaneous involution over a period of several years. Despite the inevitable regression a significant number of patients are left with unsightly fibrofatty residua or scars. More serious complications may accompany the rapid growth phase. Parents of some patients are interested in some form of active treatment, but found some conventional therapies overly aggressive.

Further background is provided by the following references each of which are incorporated herein by reference.
1. Sauder D N. J Am Acad Dermatol 2000;43:S6-11
2. Hengge et al. Lancet Infect Dis 2001;1:189-98
3. Ezekowitz R A B et al. N Engl J Med 1992;326:1456-63
4. Coughlin C M et al. Immunity 1998;9:25-34
5. Sunamura et al. Pancreas 2000;20:227-33
6. Dahl M V. J Am Acad Dermatol 2000;43:S1-5
7. Sidbury R et al. J Invest Dermatol 2000;114:770 (Abstr)
8. Takahashi K et al. J Clin Invest 1994;93:2357-64
9. Duda D G et al. Cancer Research 2000;60:1111-6
10. Imbertson L M et al. J Invest Dermatol 1998; 110: 734-39
11. Boon L M et al. J Pediatr 1996; 128:32

These references are referred to in the following section (Section IV) by their respective reference numeral.

### V. DETAILED DESCRIPTION OF INVENTION APPLIED TO INFANTILE HEMANGIOMAS AND EPITHELIOID HEMANGIOENDOTHELIOMA

The option of topical 5% imiquimod cream at a frequency of three times per week was offered to the parents of 4 patients with IH. In each case this treatment option was found acceptable by both parents, who fully understood that this was an off label use of the medication.

EH is a low grade malignant vascular tumor that occurs most commonly in the superficial or deep soft tissues of the body. The patient refused surgery for personal reasons, but acquiesced to the use of 5% imiquimod cream, even though such usage was off label.

We report here for the first time to our knowledge the apparent efficacy of topical application of the immune response modifier imiquimod in the treatment of 4 patients with infantile hemangioma (IH) and one with an epithelioid hemangioendothelioma (EH).

A. Infantile Hemangiomas

1. Case 1

A 7-month old boy presented for consultation with an IH on the frontal scalp (3.0×2.5 cm) that was noticed at age 2 months and enlarged rapidly. MRI analysis showed a soft tissue mass extending to the outer table of the skull, suggestive of compound IH. After 4 weeks of thrice weekly application of imiquimod, the lesion appeared less protuberant but exhibited marked erythema and crusting. Therapy was discontinued for 2 weeks with disappearance of inflammation and a marked reduction in the size of the IH. Then the frequency of imiquimod application was increased to every other day for only 2 weeks because of reappearance of inflammation and crusting. At follow-up examination 4 weeks later of the 10-month-old infant there was virtually complete clinical regression of the IH with return to normal skin color. At the most recent follow-up visit, the 20-month-old patient, was in excellent health with no recurrence of the lesion.

2. Case 2

A 4-month-old girl presented with a red-grey bulbous, 4.5 cm in diameter IH on the frontal scalp that appeared at 1 month of age and grew rapidly. MRI analysis supported the clinical diagnosis of IH. Topical application of imiquimod was started with a frequency of 3 times weekly that resulted after 3 weeks in marked crusting and erythema. The medication at the mother's request was discontinued. Two months later, because of rapid regrowth of the lesion, the mother returned to clinic and requested renewal of the therapy. Imiquimod was restarted and increased to every other day for 6 weeks. This course of therapy was completed despite recurrence of erythema and crusting. Four weeks later, when the patients was 9 months old, examination revealed near complete regression of the lesion. Therapy was discontinued. At last follow-up at age 16 months the lesion had completely disappeared.

3. Case 3

A 4-month old, healthy female infant was born with a small reddish, flat IH over the left lower lip that started to grow rapidly. Imiquimod, 5%, cream was applied every other day to the outer lip area only. After 2 weeks of application she developed marked inflammation and crusting over the lip area, but the medication was continued. After one and a half months of therapy most of the IH over the outer lip area had regressed. The patient is 6 months old and continues the treatment. The untreated area (the inner buccal mucosa) persisted but had also decreased in size.

4. Case 4

A 3 month old female infant was born with a IH on the right upper eyelid that grew rapidly during her first few months life. An MRI study excluded any involvement of any underling ocular structure. Topical application of imiquimod 5% cream was applied every other day for one month over the entire IH. Erythema and crusting developed in two weeks. After one month of application, the IH had regressed almost to the skin level and the patient continues treatment.

TABLE I
Hemangiomas

| | | | | Patient | | | |
|---|---|---|---|---|---|---|---|
| | | | | age at | | | |
| | | | | onset of | | | |
| | | Presentation | Location and | imiquimod | Schedule | Side effects and | Results and |
| | Sex | of lesion | depth | treatment | treatment | observations | follow up |
| Patient 1 | Male | Appeared at 2 | Frontal scalp | 7 months | 4 weeks of 3 times | Erythema with | Virtually complete clinical |
| | | months and | Cutaneous and | | weekly application | crusting during | regression |
| | | grew rapidly | subcutaneous | | Resting period of 2 | treatment | Healing without scarring and |
| | | | | | weeks | No neurological | without effecting the growth |
| | | | | | 2 weeks of every | abnormalities | of hair at 20 months of age |
| | | | | | other day | | |
| Patient 2 | female | Appeared at 1 | Frontal scalp | 4 months | 3 times weekly, for | Erythema and crusting | 9 months age there was near |
| | | month and | Cutaneous and | | 3 weeks. | during treatment | complete regression of the |
| | | grew rapidly | subcutaneous | | Resting period for 2 | Normal neurologic | lesion. |
| | | | | | months. | examination | At age 22 months there was no |
| | | | | | Then every other | | evidence of recurrence. Normal |
| | | | | | day for 6 weeks | | hairs covering the area. No |
| | | | | | | | evidence of scarring. |
| Patient 3 | female | Present at birth | Lip | 4 months | 3 times weekly for 6 | Erythema and crusting | Persistence with marked |
| | | as reddish | Cutaneous and | | weeks | during treatment | decrease in size of the IH over |
| | | macule that | subcutaneous | | | Normal neurologic | the outer lip area (treated area). |
| | | grew rapidly | | | | examination | Persistent but slight decrease in |
| | | | | | | | size of the IH in the inner buccal |
| | | | | | | | mucosa (area not treated). |
| | | | | | | | Treatment continues |
| Patient 4 | female | Present at birth | Right upper eyelid | 3 months | Every other day for | Erythema and crusting | After 1 month of treatment, the |
| | | as flat lesion | Cutaneous and | | 1 month | after 2 weeks | IH had regressed almost to the |
| | | that rapidly | subcutaneous | | | treatment. | normal plane of the skin surface. |
| | | thickened | | | | Normal neurologic | Persistence of erythema and |
| | | | | | | examination | crusting |

B. Epithelioid Hemangioendothelioma

A 52-year-old female patient developed a nodule on the left cheek that started to grow over the period of 6 months. She presented to the clinic with a 2.5 cm dusky red, firm nodule on her left cheek that on biopsy revealed a lesion consistent with an epithelioid hemangioendothelioma The patient refused surgery. However, she was willing to apply 5% imiquimod topical cream after she was informed of the success of this agent in treatment of some skin cancers and hemangiomas.

After 2 weeks of the application of imiquimod she developed erythema and crusting and the nodule decreased remarkably in size. Re-biopsy revealed persistent hemangioendothelioma but with mild atypia and marked inflammation and fibrosis. At this point, the patient agreed to have surgery, but the lesion continued to improve in the interim, developing a central cleared area. Biopsy just before surgery revealed scarring in the central cleared area and some residual tumor with marked inflammation in the peripheral inflamed rim. The surgical specimen revealed only scarring with no evidence of tumor.

C. Discussion
Imquimod—an imidazoquinoline amine—is an immune response modifier that acts by effecting both innate and acquired immune responses. The effect on innate immunity is achieved through production of a large spectrum of cytokines including, but not restricted to, IFN-α, interleukin 6 (IL-6), and tumor necrosis factor-alpha (TNF-α), as well as by enhancement of natural killer (NK) cell activity. B cell proliferation and maturation is stimulated¹ with production of IgG2a. The latter acts like analogously to the immunostimulatory CpG-sequences. These structures result in increased innate immunity². Acquired immunity is also enhanced and includes production of IL-12, resulting in an increase in cytotoxic T-lymphocytes and the release of IFN-γ.¹
IFN-α, administered through systemic means has been shown in the literature to be an effective treatment of IH.³ The exact mechanism of action is not fully understood. However, this route of administration has been associated with the occurrence of significant neurologic complications, most seriously spastic dysplegia. IFN-α, locally produced by imiquimod, may clearly be one of the active agents responsible for the regression of the IH cited in this report. The local release of IFN-α for a few months in this study is strikingly different than the systemic administration of high doses of interferon for many months that were associated with neurologic changes. Nevertheless all of our patients were examined neurologically and none showed any adverse effects. Another possible mechanism or action of imiquimod in IH may relate to the recent reports concerning the tumor suppressive and antiangiogenic effects of IL-12^{4,5}. In nude mice and rats, topical application of 1 and 5% cream has been shown to result in a local increase in IFN-α and TNF-α.⁶
In a polyoma virus-induced hemangioendothelioma model, topical imiquimod has been shown to result in increased intratumoral mast cells as well as elevated levels of tissue inhibitor of metalloproteinases type 1 (TIMP-1) and TNF-α with evidence of increased apoptosis.⁷ Increased density of mast cells and increased expression of TIMP-1 has also been reported in the involutive phase compared to proliferative phase of IH. Thus, imiquimod treatment may be causing hypothetically a recapitulation of the natural involutive process of IH.
A variety of studies in rodents, monkeys and humans using in vivo and in vitro techniques, including splenic cultures of human lymphocytes treated with imiquimod, have shown the production of other cytokines including IL-2 and IFN-γ as a result of IL-12 production.⁹ Activation of NK cells by IFN-γ has the potential to cause destruction of IH cells. IFN-γ inducible IP-10 may in turn have a direct antiangiogenic effect as has been shown in experimental tumor models.¹⁰ Clearly a variety of mechanisms may reasonably be involved in imiquimod-induced regression of IH, and further clinical and experimental studies are certainly warranted.
Finally, our cases are clearly different from the so-called “congenital hemangiomas” that are fully formed at birth and may regress over several months. All of the hemangiomas in this report grew rapidly after birth in the classical manner of IH. Thus, their rapid response to imiquimod cannot be ascribed to spontaneous involution associated with the congenital lesions.¹¹

D. Conclusions

We present a remarkable therapy for infantile hemangiomas and possibly for other vascular tumors such as epithelioid hemangioendothelioma. In the two patients who completed therapy there was complete disappearance of their lesions with no recurrence at least at one year of follow up. The other two patients continue to respond dramatically to treatment. An interesting case of a vascular tumor of borderline significance occurring in a patient who initially refused standard therapy allowed us to observe another malignant tumor in the skin that responds to this immunomodulatory drug. Stimulated by these extraordinary cases we have designed a much larger study that includes pathology documentation of the response as well as research into the mechanism of action of imiquimod.

1. A method of applying Imiquimod or equivalent substance to reduce vacular tumors or vascular lesions of the skin or mucosal sites.

2. A method of treating inflammatory lesions of the skin caused by immune regulatory mechanisms.
